(19) United States
(12) Patent Application Publication
(10) Pub. No.: US 2016/0339151 A1
(43) Pub. Date: Nov. 24, 2016

(54) HYDROGEL HAVING A DECOMPOSITION RATE CAPABLE OF BEING REGULATED IN SITU AND METHOD FOR MANUFACTURING SAME

(71) Applicant: OSSTEMIMPLANT CO., LTD., Seoul (KR)

(72) Inventors: Yong Il CHUNG, Busan (KR); Da Mi CHOI, Busan (KR); Ju Dong SONG, Busan (KR); Eun Jung KANG, Busan (KR); Tae Gwan EOM, Busan (KR); Kyoo Ok CHOI, Seoul (KR)

(21) Appl. No.: 15/227,044

(22) Filed: Aug. 3, 2016

Related U.S. Application Data
(63) Continuation-in-part of application No. 14/367,906, filed on Jun. 21, 2014, now abandoned.

(51) Int. Cl. A61L 27/58; A61L 27/24; A61L 27/22; B29C 39/00; A61L 27/26; A61L 27/52; B29C 35/08; B29B 7/88; A61L 27/18; A61L 27/20

(52) U.S. Cl. CPC A61L 27/58; A61L 27/18; A61L 27/24; A61L 27/227; A61L 27/222; A61L 27/20; A61L 27/26; A61L 27/52; B29C 35/08; B29B 7/88; B29C 39/003; A61L 2430/02; A61L 2430/40; B29K 2071/02

(57) ABSTRACT

A hydrogel and a method for manufacturing the same. The hydrogel, which can be freely used during treatment irrespective of the shape of a bone defect region, has a decomposition rate capable of being regulated in situ and can thus be rapidly decomposed over a certain time, i.e., after the completion of bone regeneration. Furthermore, a hydrogel membrane using the hydrogel which is applied to a bone defect region irrespective of the shape thereof, and a method for manufacturing the same.

### CROSS-REFERENCE TO RELATED APPLICATION(S)

This application is a continuation-in-part of U.S. patent application Ser. No. 14/367,906, filed Jun. 21, 2014, which was the National Stage of International Application No. PCT/KR2012/008017, filed Oct. 4, 2012, which claimed priority to Korean Patent Application No. 10-2011-0139849, filed Dec. 22, 2011, the disclosure of which is incorporated in their entireties herein by reference.

### BACKGROUND OF THE INVENTION

1. Field of the Invention

The present invention relates to a hydrogel with adjustable biodegradation rate and a method for preparing the same and, more particularly, to a hydrogel which can be rapidly degraded due to its adjustable biodegradation rate after a certain time, i.e., after completion of bone regeneration, and a method for preparing the same. Moreover, the present invention relates to a hydrogel membrane which can be freely applied to a bone defect regardless of the shape of the bone defect and a method for preparing the same.

2. Description of Related Art

Sheet-type dental membranes have been mainly used in the past, but these sheet-type dental membranes should be cut to fit the shape of a bone defect, and thus the total time of surgical procedure is increased. Moreover, in the functional aspect, the sheet-type membrane is mainly composed of collagen and thus is rapidly absorbed into the body, and thus its space maintaining and soft tissue shielding functions deteriorate.

To solve these problems, U.S. Pat. No. 6,306,922 discloses photopolymerizable hydrogel membranes, but these photopolymerizable hydrogel membranes have very low biodegradability due to high physical strength.

That is, in the case of the dental membrane, the membrane strength should be maintained until bone regeneration is complete and should be rapidly degraded after the completion of bone regeneration such that the membrane does not act as a barrier to the fusion of hard tissue and soft tissue. However, the photopolymerizable hydrogel membranes cannot be degraded at an appropriate time due to high physical strength.

### SUMMARY OF THE INVENTION

Accordingly, the present invention has been made to solve the above-described problems, and an object of the present invention is to provide a hydrogel which can be rapidly degraded due to its adjustable biodegradation rate after a certain time, after completion of bone regeneration, and a method for preparing the same. Moreover, another object of the present invention is to provide a hydrogel membrane which can be freely applied to a bone defect regardless of the shape of the bone defect and a method for preparing the same so as to overcome one inconvenience in the use of conventional sheet-type membranes.

To achieve the above objects, the present invention provides a hydrogel and a hydrogel membrane, each comprising polyethylene glycol (PEG) having a photopolymerizable functional group; a natural polymer additive forming an interpenetrating polymer network (IPN) together with photopolymerized PEG and having a viscosity and a biodegradation rate higher than those or the PEG; and a photopolymerization initiator generating a radical for initiating photopolymerization of the PEG.

Moreover, the present invention provides a method for preparing a hydrogel, the method comprising the steps of: i) preparing a first liquid containing polyethylene glycol (PEG) having a photopolymerizable functional group and a second liquid containing a natural polymer additive having a viscosity and a biodegradation rate higher than those of the PEG and a photopolymerization initiator generating a radical for initiating photopolymerization of the PEG; ii) mixing the prepared first and second liquids in a cavity; and iii) forming a hydrogel in the form of an interpenetrating polymer network (IPN) by irradiating visible light to the mixed solution.

Furthermore, the present inversion provides a method for preparing a hydrogel membrane, the method comprising the steps of: i) preparing a first liquid containing polyethylene glycol (PEG) having a photopolymerizable functional group and a second liquid containing a natural polymer additive having a viscosity and a biodegradation rate higher than those of the PEG and a photopolymerization initiator generating a radical for initiating photopolymerization of the PEG; ii) mixing the prepared first and second liquids in a cavity; iii) shaping the mixed solution in the form of a membrane; and iv) forming a hydrogel in the form of an interpenetrating polymer network (IPN) by irradiation, with visible light to the mixed solution.

In addition, the present invention provides a method for preparing a hydrogel membrane, the method comprising the steps of: i) preparing a first liquid containing polyethylene glycol (PEG) having a photopolymerizable functional group and a second liquid containing a natural polymer additive having a viscosity and a biodegradation rate higher than those of the PEG and a photopolymerization initiator generating a radical for initiating photopolymerization of the PEG; ii) mixing the prepared first and second liquids in a cavity; iii) forming a hydrogel in the form of an interpenetrating polymer network (IPN) by irradiating visible light to the mixed solution; and iv) shaping the hydrogel in the form of a membrane.

The PEG may have a chemical formula of (—CH2CH2O—) n (n is an integer of 10 to 1,000) and comprise linear (2-arm) PEG; branded (4 or 8-arm) PEG, star-shaped (multi-arm) PEG or a combination thereof within the above range of the molecular weight.

Moreover, the photopolymerizable functional group may comprise at least one selected from the group consisting of acrylate, methacrylate, coumarin, thymine, and cinnamate.

The natural polymer additive may comprise at least one selected from the group consisting of carboxyl methyl cellulose, heparan, sulfate, hyaluronic acid, collagen, chitosan, dextran, and alginate, and the molecular weight of the natural polymer additive may preferably be 100,000 to 10,000,000. Moreover, the photopolymerization initiator may generate a radical in response to visible light of a wavelength of 400 to 750 nm.

The dental hydrogel of the present invention can be freely applied to a bone defect regardless of the shape of the bone defect and has an interpenetrating polymer network (IPN) formed by PEG and a natural polymer additive, which makes it possible to adjust its biodegradation rate such that it can maintain the space maintaining and soft tissue shielding functions until a certain time, i.e., until bone regeneration is complete and can be rapidly degraded after the completion of bone regeneration.

### BRIEF DESCRIPTION OF THE DRAWINGS

FIG. 1 is a conceptual diagram showing the formation and degradation of a hydrogel according to an embodiment of the present invention.

FIG. 2 is a schematic diagram showing the mixing of a first liquid containing PEG and a second containing a natural polymer additive and a photopolymerization initiator in a cavity.

FIG. 3 shows the degradation rates of each sample prepared according to Tables 1 to3.

FIG. 4 shows the results of compressive fracture strength (in vitro).

FIG. 5 shows the photo image for flowability test for various samples (M: a reference liquid sample, of which a movement distance is 0.2 cm/sec).

FIG. 6 shows the results of flowability (in vitro).

FIG. 7 shows (A) an implant grafted in the canine mandibular bone after induction of dehescience defect, (B) a photograph showing application of a bone graft material, and (C) a photograph showing application of a hydrogel membrane.

### DETAILED DESCRIPTION OF THE INVENTION

Hereinafter, a hydrogel and a method for preparing the same in accordance with an aspect of the present invention will be described in detail with reference to the accompanying drawings.

A hydrogel of the present invention comprises polyethylene glycol (PEG) having a photopolymerizable functional group, a natural polymer additive forming an interpenetrating polymer network (IPN) together with photopolymerized PEG and having a viscosity and a biodegradation rate higher than those of the PEG, and a photopolymerization initiator generating a radical for initiating photopolymerization of the PEG, and has the interpenetrating polymer network (IPN) formed by mixing photopolymerized PED and the natural polymer additive.

A hydrogel using photopolymerization, which is used in a conventional membrane, has a very dense polymer network structure, in which several PEGs are cross-linked on one connecting vertex, and exhibits high physical strength. As a result, the conventional hydrogel is not degraded after completion of bone regeneration but remains in the body, and thus the membrane itself acts as a barrier to the fusion of hard tissue and soft tissue.

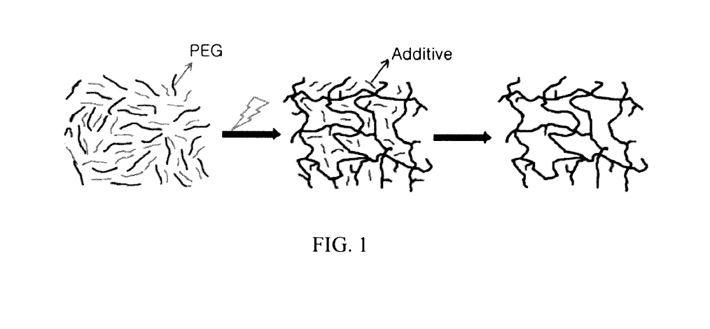

However, as shown in FIG. 1, in the case of a membrane to which the hydrogel of the present invention is applied, a natural polymer additive that forms the interpenetrating polymer network is first degraded at an appropriate time (target point), and thus the internal network structure of the hydrogel becomes loose. As a result, immune cells can easily infiltrate into the loose network, and thus the membrane is rapidly degraded.

That is, the natural polymer additive having a biodegradation rate higher than that of PEG leads the biodegradability, which thus increases the biodegradation rate of the hydrogel. Moreover, the addition of a natural polymer increases the affinity between the hydrogel and tissue, and thus when the hydrogel is used as a dental membrane, the fusion of hard tissue and soft tissue can be promoted.

The natural polymer additive may include various natural polymer materials having a biodegradation rate higher than that of PEG and may preferably include at least one selected from the group consisting of carboxyl methyl cellulose, heparan sulfate, hyaluronic acid, collagen, chitosan, dextran, and alginate. Here, the molecular weight of the natural polymer additive may preferably be in the range of 100,000 to 10,000,000.

Meanwhile, the PEG that forms the backbone of the polymer network is a polymer having a chemical formula of (—CH2CH2O—)n (n is an integer of 10 to 1,000), and linear (2-arm) PEG, branded (4 or 8-arm) PEG, and star-shaped (multi-arm) PEG may be used alone or in a combination thereof within the above range of the molecular weight.

The PEG component may have various photopolymerizable functional groups and may preferably have at least one functional group selected from the group consisting of acrylate, methacrylate, coumarin, thymine, and cinnamate.

Moreover, the photopolymerization initiator serves to generate a radical for initiating photopolymerization of the PEG and may preferably generate a radical in response to visible light of a wavelength of 400 to 750 nm.

The hydrogel may be prepared by the steps of i) preparing a first liquid containing PEG and a second containing a natural polymer additive and a photopolymerization initiator; ii) mixing the prepared first and second liquids in a cavity and iii) forming a hydrogel in the form of an interpenetrating polymer network (IPN) by irradiating visible light to the mixed solution.

Further, the membrane using the hydrogel may be prepared by the steps of i) preparing a first liquid containing PEG and a second containing a natural polymer additive and a photopolymerization initiator; ii) mixing the prepared first and second liquids in a cavity; iii) shaping the mixed solution in the form of a membrane; and iv) applying visible light to the membrane, or by the steps of: i) preparing a first liquid containing PEG and a second containing a natural polymer additive and a photopolymerization initiator; ii) mixing the prepared first and second liquids in a cavity; iii) forming a hydrogel in the form of an interpenetrating polymer network (IPN) by irradiation with visible light to the mixed solution; and iv) shaping the hydrogel in the form of a membrane.

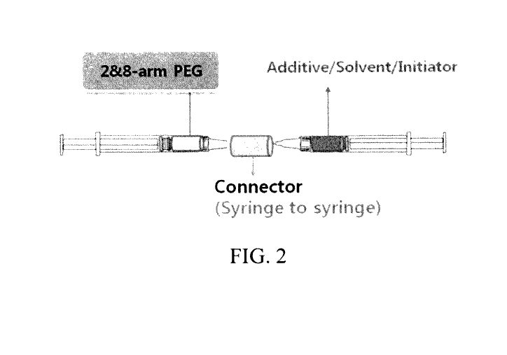

In detail, as shown in FIG. 2, the method for preparing a hydrogel according to the present invention comprises the steps of: preparing a first liquid containing PEG and a second containing a natural polymer additive and a photopolymerization initiator; and mixing the prepared first and second liquids in a cavity. Here, the natural polymer additive imparts viscosity to the mixed solution to prevent the mixed solution from flowing to the periphery, and thus it is preferable that the natural polymer additive has a viscosity higher than that of PEG.

Since the mixed solution has a viscosity sufficient to prevent it from flowing to the periphery, it is possible to freely shape the mixed solution in the form of an appropriate membrane by applying the mixed solution to a mold corresponding to the shape of a bone defect using an injectable container, etc., or by directly applying the mixed solution to the bone defect of a mammalian bone defect. The thus shaped membrane is polymerized by irradiation with visible light to finally form a hydrogel membrane in the form of an interpenetrating polymer network (IPN).

For the use of the hydrogel of the present invention as a dental material, various methods such as a method of using a paste container, a direct shaping method, a method of using a mold, etc. may be used in addition to the above-mentioned method of using an injectable container without departing from the gist of the present invention.

### EXAMPLE 1

### Preparation of Hydrogel

In order to determine a hydrogel composition having the optimum performance and physical properties, a variety of hydrogels were prepared while changing the composition and content of PEG according to the kinds of natural polymer additive and photopolymerization initiator as follows.

In experimental group 1, as shown in Table 1 below, a mixture of collagen and hyaluronic acid (HA) (10:4 w/w) was used as a natural polymer additive, and DPO (diphenyl(2,4,6-trimethylbenzoyl) phosphine oxide) was used as a photopolymerization initiator. In addition, as PEG, 2-arm PEG alone, 8-arm PEG alone or a mixture of 2-arm PEG and 8-arm PEG was used in varying amounts (wt %), and as a solvent, deionized (D.I.) water) was used so that the total weight of each sample would reach 100 wt %.

TABLE 1

| | Sample No. | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| 2-arm | 40 | 30 | 20 | 10 | — | — | — | — | 20 | 15 | 10 | 5 |
| PEG | | | | | | | | | | | | |
| 8-arm | — | — | — | — | 40 | 30 | 20 | 10 | 20 | 15 | 10 | 5 |
| PEG | | | | | | | | | | | | |
| Collagen | 1.0 | 1.0 | 1.0 | 1.0 | 1.0 | 1.0 | 1.0 | 1.0 | 1.0 | 1.0 | 1.0 | 1.0 |
| HA | 0.4 | 0.4 | 0.4 | 0.4 | 0.4 | 0.4 | 0.4 | 0.4 | 0.4 | 0.4 | 0.4 | 0.4 |
| DPO | 0.1 | 0.1 | 0.1 | 0.1 | 0.1 | 0.1 | 0.1 | 0.1 | 0.1 | 0.1 | 0.1 | 0.1 |
| Ethanol | 5 | 5 | 5 | 5 | 5 | 5 | 5 | 5 | 5 | 5 | 5 | 5 |

In experimental group 2, as shown in Table 2 below, gelatin was used as a natural polymer additive, and DPO was used as a photopolymerization initiator. In addition, the kind and amount of PEG used were changed in the same manner as described for experimental group 1.

TABLE 2

| | Sample No. | | | | | | | | | | | |
|---|---|---|---|---|---|---|---|---|---|---|---|---|
| | 13 | 14 | 15 | 16 | 17 | 18 | 19 | 20 | 21 | 22 | 23 | 24 |
| 2-arm | 40 | 30 | 20 | 10 | — | — | — | — | 20 | 15 | 10 | 5 |
| PEG | | | | | | | | | | | | |
| 8-arm | — | — | — | — | 40 | 30 | 20 | 10 | 20 | 15 | 10 | 5 |
| PEG | | | | | | | | | | | | |
| Gelatin | 0.4 | 0.4 | 0.4 | 0.4 | 0.4 | 0.4 | 0.4 | 0.4 | 0.4 | 0.4 | 04 | 0.4 |
| DPO | 0.1 | 0.1 | 0.1 | 0.1 | 0.1 | 0.1 | 0.1 | 0.1 | 0.1 | 0.1 | 0.1 | 0.1 |
| Ethanol | 5 | 5 | 5 | 5 | 5 | 5 | 5 | 5 | 5 | 5 | 5 | 5 |

In experimental group 3, as shown in Table 3 below, carboxymethyl cellulose (CMC) was used as a natural polymer additive, and Eosin Y was used as a photopolymerization initiation. In addition, the kind and amount of PEG used were changed in the same manner as described for experimental groups 1 and 2 above.

TABLE 3

| | Sample No. | | | | | | | | | | | |
|---|---|---|---|---|---|---|---|---|---|---|---|---|
| | 25 | 26 | 27 | 28 | 29 | 30 | 31 | 32 | 33 | 34 | 35 | 36 |
| 2-arm | 40 | 30 | 20 | 10 | — | — | — | — | 20 | 15 | 10 | 5 |
| PEG | | | | | | | | | | | | |
| 8-arm | — | — | — | — | 40 | 30 | 20 | 10 | 20 | 15 | 10 | 5 |
| PEG | | | | | | | | | | | | |
| CMC | 0.5 | 0.5 | 0.5 | 0.5 | 0.5 | 0.5 | 0.5 | 0.5 | 0.5 | 0.5 | 0.5 | 0.5 |
| Eosin Y | 0.1 | 0.1 | 0.1 | 0.1 | 0.1 | 0.1 | 0.1 | 0.1 | 0.1 | 0.1 | 0.1 | 0.1 |

### EXAMPLE 2

### Degradation Test

Among resorbable membranes, collagen membranes that are most widely used have a problem in that they have an excessively high in viva degradation rate, and thus cannot maintain their function as membranes up to the time point of bone formation. For example, commercially available Bio-Gide (Gistlich) shows a degradation rate of about 48% at a time point of 96 hours in a degradation test. Thus, in the present invention, a degradation test was performed in order to select a hydrogel membrane having excellent in vivo degradability compared to conventional collagen membranes from among hydrogel membranes (sample Nos. 1 to 36) having the compositions shown in Tables 1 to 3.

Because it is required that hydrogel membranes have low in vivo degradation rate in an initial stage and are completely degraded in vivo after a certain time, primary selection was performed based on a degradation rate of 48% at a time point of 120 hours.

To evaluate the degradation rate of hydrogel membranes, using an incubation device equipped with a stirrer, hydrogels were immersed in PBS containing 0.5 mg of sodium azide at 37° C., and the initial sample weight (Mo) and the sample weight after freeze-drying were measured several times, thereby determining the degradation distribution of each hydrogel.

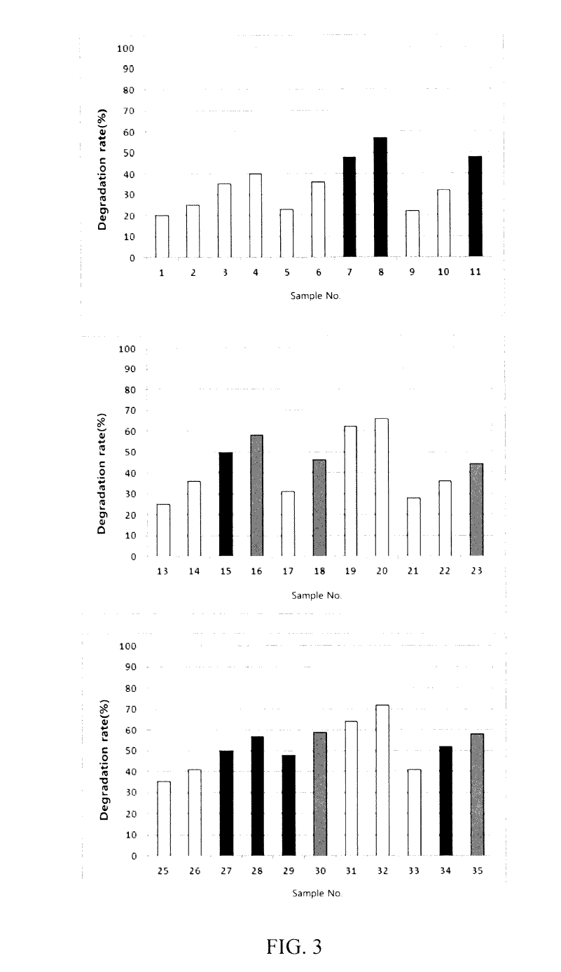

The results of measuring the degradation rate of each sample are shown in FIG. 3 and summarized in Table 4 below. Samples having a degradation rate higher than 48±11% were excluded from a subsequent test, and a subsequent evaluation test was performed only on samples having a degradation rate of 48±11% or lower at a time point of 120 hours.

TABLE 4

| Degradation | | Bar color |
|---|---|---|
| Rate (%) | Sample No. | (@ FIG. 3) |
| 48 ± 5% | 7, 8, 11, 15, 27, 28, 29, 34 | black |
| 48 ± 7% | 16, 18, 30, 35 | dark gray |
| 48 ± 9% | 56 | gray |
| 48 ± 11% | 19, 23, 26, 33, | neutral tint |
| Others | Excluded from subsequent test | white |

### EXAMPLE 3

### Measurement of Compressive Fracture Strength (In Vitro)

In order to evaluate the physical properties of hydrogel membranes, the durability of hydrogel membranes against external impact was measured. Hydrogel membranes were formed into a cylindrical shape having a diameter of 10 mm and a height of 3 mm, and the durability thereof was measured under the measurement conditions shown in Table 5 below.

After a hydrogel membrane is applied to an affected part and gingiva is finally sutured, the patient should be capable of resisting an external physical force applied to the membrane during the healing period. For this reason, in order for the function of hydrogel membranes to be perfectly performed, a compressive strength of a certain level or higher is an important factor in evaluation of the physical properties of hydrogel membranes.

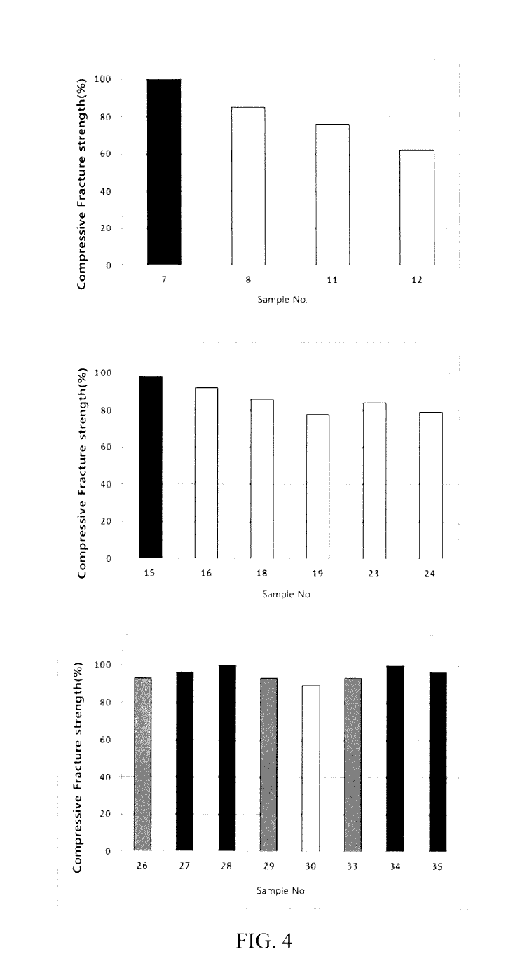

The results of measuring the durability of samples primarily selected in Example 2 are shown in FIG. 4 and summarized in Table 6 below. A subsequent evaluation test was performed only on samples showing ±9% relative to the highest fracture strength.

TABLE 5

| | Value | Unit |
|---|---|---|
| Break Sensitivity | 20 | % |
| Break Threshold | 2.240 | N |
| Data Acq. Rate | 10.0 | Hz |
| Pre-Load | 44.482 | N |
| Pre-Load Speed | 2.540 | mm/mm |
| Strain Endpoint | 0.100 | mm/min |
| Test Speed | 0.50 | mm/min |

TABLE 6

| Percentage relative | | |
|---|---|---|
| to highest compressive | | Bar color |
| fracture strength | Sample No. | (@ FIG. 4) |
| ±5% | 7, 15, 27, 28, 34, 35 | black |
| ±7% | 26, 29, 33 | dark gray |
| ±9% | 16 | gray |
| Other | Excluded from test | white |

### EXAMPLE 4

### Evaluation of Flow Properties (In Vitro)

As shown in FIG. 2, the hydrogel membrane according to the present invention is a 2-syringe type membrane divided into a syringe portion that provides PEG and a syringe portion that provides a natural polymer additive. Immediately before use, the user connects the syringes to each other by a connector. Then, the contents in the syringes are mixed in a syringe-to-syringe manner and applied to an affected part. Thus, the hydrogel membrane is a free-type membrane.

If the mixed hydrogel membrane has low viscosity, when it is applied to an affected part, it will flow to the surrounding portion to reduce the user's convenience. Particularly, the hydrogel membrane will penetrate a bone graft material to interfere with bone formation. On the contrary, if the mixed hydrogel membrane has high viscosity, an excessive ejection force should be applied to a plunger to reduce the user's convenience, and the cohesive force between thickener molecules will increase so that the hydrogel membrane will show poor spreading properties around a bone graft material. In addition, the adhesion between alveolar bone and the hydrogen membrane will be reduced.

Thus, only when the hydrogel membrane ensures the optimum flow properties (i.e., suitable viscosity), the hydrogel membrane will not penetrate a bone graft material and, at the same time, will show good spreading properties around a bone graft material so as to have high adhesion, and can also show excellent properties in terms of reducing the mobility of a bone graft material and maintaining a space.

Such flow properties were measured on the samples showing ±9% relative to the highest compressive fracture strength in Example 3.

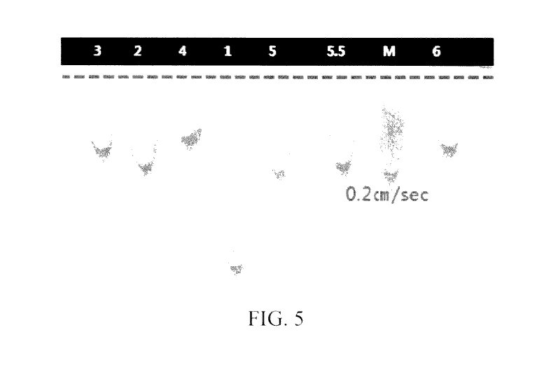

Artificial bone models having a uniform surface were laid horizontally. In this state, each of the bone models was injected with 0.2 cc of each of hydrogel membrane samples. Then, the artificial bone models were stood vertically, and the distance by which each sample moved for the same time was measured. As a control, a reference liquid having a viscosity corresponding to a movement distance of 0.2 cm per second was used to compare flowability (see FIG. 5).

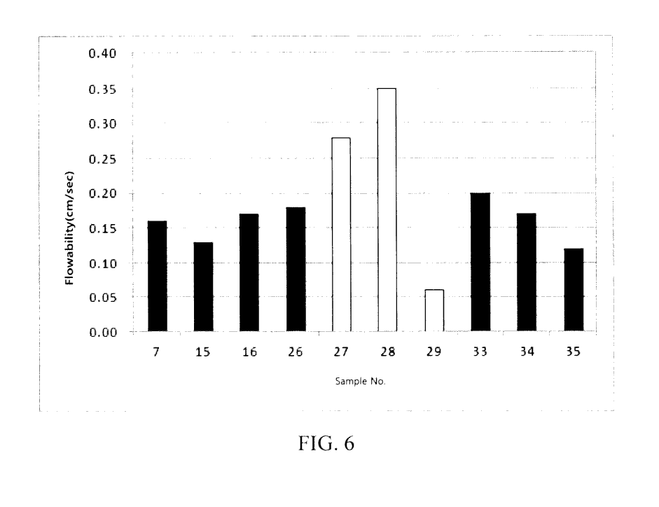

As can be seen from the results in FIGS. 5 and 6, the results of comparing flowability with that of the control reference liquid indicated that, when the flow distance per second is 0.1 cm or less, problems can arise, such as excessive ejection force and adhesive properties as mentioned above, and when the flow distance per second is 0.2 cm or more, the hydrogel membrane can penetrate a bone graft material to interfere with bone formation. For these reasons, a flow distance ranging from 0.1 to 0.2 cm/sec was selected, and based on this, sample Nos. 7, 15, 16, 26, 33, 34 and 35 were selected.

### EXAMPLE 5

### Animal Study (In Vivo)

In order for the hydrogel membrane of the present invention to be used as a membrane for induction of periodontal tissue regeneration to perform effective GBR, the hydrogel membrane should effectively act for bone formation while functioning as a membrane to block penetration of soft tissue. In addition, a grafted hydrogel membrane should have biological properties such that it does not cause inflammation in the surrounding tissue and, at the same time, is degraded and completely absorbed after completion of bone formation.

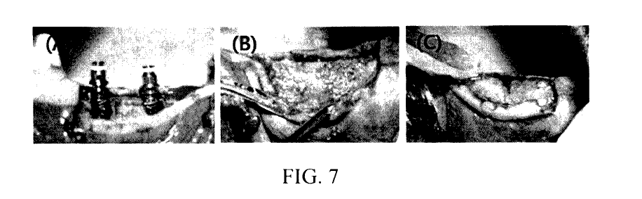

Thus, in order to examine the biological properties (including effectiveness) of sample Nos. 7, 15, 16, 26, 33, 34 and 35 selected in Example 4 above, an in vivo experiment in animals was performed as shown in FIG. 7.

FIG. 7 shows (A) an implant grafted in the canine mandibular bone after induction of dehescience defect, (B) a photograph showing application of a bone graft material, and (C) a photograph showing application of a hydrogel membrane.

As shown in FIG. 7, at 12 weeks after application of each hydrogel membrane, tissue was collected from each test animal, and a tissue slide was prepared therefrom. Each of the tissue slides was histopathologically examined, and the results of quantitative evaluation for the test items shown in Table 7 were scored, and the total score of each sample was calculated. The results of the calculation are summarized in Table 8 below.

TABLE 7

| | | Inflam- | | |
|---|---|---|---|---|
| Blocking | Bone formation | matory | Degradation | |
| ability | ability | reaction | rate | Score |
| 100% | 85% or more relative | Absent/ | 3.0-50% | 3 |
| blocked | to normal bone | minimal | | |
| 80% | 75% or more relative | Mild | 20-30, 50-60% | 2 |
| blocked | to normal bone | | | |
| 60% | 65% or more relative | Moderate | 10-20, 60-70% | 1 |
| blocked | to normal bone | | | |
| 40% | 55% or more relative | Severe/ | 10% or less, | 0 |
| blocked | to normal bone | Marked | 70% or more | |

TABLE 8

| Evaluation | Sample No. | | | | | | |
|---|---|---|---|---|---|---|---|
| item | 7 | 15 | 16 | 26 | 33 | 34 | 35 |
| Blocking | 2 | 2 | 2 | 3 | 3 | 2 | 2 |
| ability | | | | | | | |
| Bone | 2 | 2 | 2 | 3 | 3 | 3 | 2 |
| formation | | | | | | | |
| ability | | | | | | | |
| Inflammatory | 3 | 1 | 2 | 2 | 2 | 3 | 2 |
| reaction | | | | | | | |
| Degradation | 1 | 2 | 2 | 1 | 2 | 3 | 2 |
| ability | | | | | | | |
| Sum | 8 | 7 | 8 | 9 | 10 | 11 | 8 |

In the results of Examples 2 to 5, when the DPO photopolymerization initiator was applied to the same kind of PEG, it showed a tendency to form a hydrogel membrane having a relatively high strength, suggesting that when the hydrogel membrane is transplanted in vivo, it will have a slow degradation rate and cannot be easily degraded at a suitable time point (i.e., after completion of bone formation). In addition, when the mixture of collagen and HA was used as the natural polymer additive, it was observed that the degradation of the polymer was lower than that of gelatin or CMC.

When gelatin was used as the natural polymer additive, the inflammatory reaction was relatively severe. Thus, the use of gelatin was not preferable in terms of inflammation reaction, even though the in vivo degradation rate thereof was better than that of the mixture of collagen and HA.

Because 2-PEG is a linear molecule, it has low flexibility and elasticity and high strength properties after photopolymerization. However, when Eosin Y was used instead of DPO as the photopolymerization initiator, a hydrogel membrane having excellent flexibility and elasticity properties could be prepared, because the wavelength of light absorbed became narrower so that rapid photo-curing could be prevented. However, in this case, the degradability showed a tendency to decrease, when the content of PEG increased to 30% or more.

Because the photopolymerization initiator Eosin Y absorbs blue light in a narrow wavelength range, the time taken for photopolymerization upon light irradiation was longer than that in the use of DPO. However, in this case, rapid photo-curing did not occur, and thus most of the samples excellent elasticity and flexibility properties, Particularly, the samples comprising the mixture of 2-PEG and 8-PEG showed the best hydrogel membrane characteristics.

Namely, in the case in which branched 8-PEG and linear 2-PEG formed a PEG network having an interpenetrating structure and in which CMC showing a high degradation rate in an initial stage was used as the natural polymer additive (sample Nos. 33 and 34), it could be seen that the hydrogel membrane could be degraded quickly after completion of bone formation, and thus had excellent characteristics.

Meanwhile, in another embodiment of the present invention, the hydrogel membrane may be prepared by curing the mixed solution of the first liquid and the second liquid in the form of a hydrogel by direct irradiation of visible light and then applying the hydrogel to a bone defect at an appropriate pressure.

The present invention is not limited to the above-described specific embodiments and description, and it will be appreciated by those skilled in the art that changes may be made in these embodiments without departing from the principles and spirit of the invention, the scope of which is defined in the appended claims and their equivalents.

What is claimed is:

1. A hydrogel membrane comprising:
a polyethylene glycol (PEG) having a photopolymerizable functional group;
a natural polymer additive that forms an interpenetrating polymer network (IPN) with a photopolymerized PEG and has a viscosity and in vivo degradation rate higher than those of the PEG; and
a photopolymerization initiator that generates a radical for initiating photopolymerization of the PEG.

2. The hydrogel membrane of claim 1, wherein the PEG has a formula of (—CH₂CH₂O—)ₙ (wherein n denotes molecular weight and is an integer ranging from 10 to 1,000), and comprises linear (2-arm) PEG, branched (8-arm) PEG or a mixture thereof within said molecular weight range.

3. The hydrogel membrane of claim 1, wherein the photopolymerizable functional group comprises at least one selected from among acrylate, methacrylate, coumarin, thymine and cinnamate.

4. The hydrogel membrane of claim 1, wherein the natural polymer additive comprises at least one from the group consisting of carboxymethyl cellulose, hyaluronic acid, collagen, gelatin, and combinations thereof.

5. The hydrogel membrane of claim 4, wherein the natural polymer additive has a molecular weight of 100,000-10,000,000.

6. The hydrogel membrane of claim 1, which is applied to a bone defect.

7. A method for preparing a hydrogel membrane, the method comprising:
i) preparing a first composition containing a polyethylene glycol (PEG) having a photopolymerizable functional group, and a second composition containing: a natural polymer additive having a viscosity and biodegradation rate higher than those of the PEG; and a photopolymerization initiator that generates a radical for initiating photopolymerization of the PEG;
ii) mixing the prepared first composition and second composition with each other to obtain a mixture solution; and
iii) irradiating the mixture solution with visible light to thereby form a hydrogel membrane having an interpenetrating polymer network (IPN) structure.

8. The method of claim 7, wherein the photopolymerization initiator generates a radical in response to visible light having a wavelength of 400-750 nm.

9. The method of claim 7, wherein the PEG has a formula of (—CH₂CH₂O—)ₙ (wherein n denotes molecular weight and is an integer ranging from 10 to 1,000), and comprises linear (2-arm) PEG, branched (8-arm) PEG or a mixture thereof within said molecular weight range.

10. The method of claim 7, wherein the photopolymerizable functional group comprises at least one selected from among acrylate, methacrylate, coumarin, thymine and cinnamate.

11. The method of claim 7, wherein the natural polymer additive comprises at least one from the group consisting of carboxymethyl cellulose, hyaluronic acid, collagen, gelatin, and combinations thereof.

12. The method of claim 7, wherein the natural polymer additive has a molecular weight of 100,000-10,000,000.
